(19) United States
(12) Patent Application Publication
(10) Pub. No.: US 2018/0235686 A1
(43) Pub. Date: Aug. 23, 2018

(54) SYSTEMS AND METHODS FOR ABLATION STATUS MONITORING AND CUSTOM ABLATION SHAPING

(71) Applicant: Innoblative Designs, Inc., Chicago, IL (US)

(72) Inventors: Alan V. Sahakian, North Chicago, IL (US); Yearnchee C. Wang, Mill Creek, WA (US); Terence Chan, Diamond Bar, CA (US)

(21) Appl. No.: 15/902,398

(22) Filed: Feb. 22, 2018

Related U.S. Application Data
(60) Provisional application No. 62/537,219, filed on Jul. 26, 2017.
(60) Provisional application No. 62/462,529, filed on Feb. 23, 2017.

(51) Int. Cl. A61B 18/14

(52) U.S. Cl. CPC A61B 18/14; A61B 2017/00022

(57) ABSTRACT

The invention is a system for monitoring and controlling tissue ablation. The system includes a controller configured to selectively control energy emission from an electrode array of an ablation device based on ablation feedback received during an ablation procedure with the ablation device. The controller is configured to receive feedback data from one or more sensors during the ablation procedure, the feedback data comprising one or more measurements associated with at least one of operation of the electrode array of the ablation device and tissue adjacent to the electrode array. The controller is further configured to generate an ablation pattern for controlling energy emission from the electrode array of the ablation device in response to the received feedback data.

### CROSS-REFERENCE TO RELATED APPLICATIONS

This application claims the benefit of, and priority to, U.S. Provisional Application No. 62/462,529, filed Feb. 23, 2017, and U.S. Provisional Application No. 62/537,219, filed Jul. 26, 2017, the contents of each of which are hereby incorporated by reference herein in their entireties.

### GOVERNMENT SUPPORT

This invention was made with government support under IIP 1622842 awarded by the National Science Foundation. The government has certain rights in the invention.

### FIELD

The present disclosure relates generally to medical devices, and, more particularly, to system for monitoring and controlling an ablation device to cause the ablation device to emit energy in a desired shape or pattern so as to deliver treatment for the ablation and destruction of a targeted portion of marginal tissue around the tissue cavity.

### BACKGROUND

Cancer is a group of diseases involving abnormal cell growth with the potential to invade or spread to other parts of the body. Cancer generally manifests into abnormal growths of tissue in the form of a tumor that may be localized to a particular area of a patient's body (e.g., associated with a specific body part or organ) or may be spread throughout. Tumors, both benign and malignant, are commonly treated and removed via surgical intervention, as surgery often offers the greatest chance for complete removal and cure, especially if the cancer has not spread to other parts of the body. Electrosurgical methods, for example, can be used to destroy these abnormal tissue growths. However, in some instances, surgery alone is insufficient to adequately remove all cancerous tissue from a local environment.

For example, treatment of early stage breast cancer typically involves a combination of surgery and adjuvant irradiation. Unlike a mastectomy, a lumpectomy removes only the tumor and a small rim (area) of the normal tissue around it. Radiation therapy is given after lumpectomy in an attempt to eradicate cancer cells that may remain in the local environment around the removed tumor, so as to lower the chances of the cancer returning. However, radiation therapy as a post-operative treatment suffers various shortcomings. For example, radiation techniques can be costly and time consuming, and typically involve multiple treatments over weeks and sometimes months. Furthermore, radiation often results in unintended damage to the tissue outside the target zone. Thus, rather than affecting the likely residual tissue, typically near the original tumor location, radiation techniques often adversely affect healthy tissue, such as short and long-term complications affecting the skin, lungs, and heart.

Accordingly, such risks, when combined with the burden of weeks of daily radiation, may drive some patients to choose mastectomy instead of lumpectomy. Furthermore, some women (e.g., up to thirty percent (30%)) who undergo lumpectomy stop therapy before completing the full treatment due to the drawbacks of radiation treatment. This may be especially true in rural areas, or other areas in which patients may have limited access to radiation facilities.

### SUMMARY

Tumors, both benign and malignant, are commonly treated and destroyed via surgical intervention, as surgery often offers the greatest chance for complete removal and cure, especially if the cancer has not metastasized. However, after the tumor is destroyed, a hollow cavity may remain, wherein tissue surrounding this cavity and surrounding the original tumor site can still leave abnormal or potentially cancerous cells that the surgeon fails, or is unable, to excise. This surrounding tissue is commonly referred to as “margin tissue” or “marginal tissue”, and is the location within a patient where a reoccurrence of the tumor may most likely occur.

Some alternative treatments to using radiation therapy include the use of ablation devices to be inserted within cavitary excisional beds and deliver radiofrequency (RF) energy to marginal tissue surrounding the cavity following the procedure. For example, one type of proposed ablation applicator includes a long rigid needle-based electrode applicator for delivery of RF energy to marginal tissue upon manual manipulation by a surgeon or operator. Another type of ablation application includes an umbrella-type array of electrodes jointly connected to one another and deployable in an umbrella-like fashion to deliver RF energy.

While current ablation devices may provide some form tissue ablation, none have proven to meet all needs and circumstances encountered when performing marginal cavity tissue ablation. For example, when performing an RF ablation procedure on heterogeneous tissue, such as breast tissue, electrical current generally travels through the least resistant pathway, which may result in non-uniform heating of the tissue. For example, in breast tissue, fat is interspersed with fibroglandular tissue, wherein each has a specific electrical conductivity. Generally, the greater the fat content, the greater the electrical resistance, thereby dampening the effect of electrical current emitted into the breast tissue during an RF ablation procedure, which may result in non-uniform heating of the intended marginal tissue and thus further lead incomplete ablation. Further complicating this is the fact that breast tissue density varies from patient to patient due to age and body mass index, which further adds complexity to the achieving uniform and predication ablation across a variety of patients. Furthermore, in certain instances, it may be desirable to create a non-uniform ablation within a tissue cavity. In some instances, vital organs or critical internal/external structures (e.g., bone, muscle, skin, etc.) may be in close proximity to a tissue cavity and any unintended exposure to RF energy could have a negative impact.

Current RF ablation devices are unable to provide precise control over the emission of RF energy such that they lack the ability to effectively provide transmission of energy and subsequent uniform heating throughout heterogeneous tissue during the ablation procedure. Current RF ablation devices further lack the ability to prevent emission from reaching vital organs or important internal/external structures. In particular, the long rigid needle-based electrode RF applicators generally require the surgeon or operator to manually adjust needle locations, and possibly readjust several electrodes multiple times, in order to control an ablation, which may lead to inaccuracy and difficulty in directing RF emission. The umbrella array RF applicators are limited by their physical geometry, in that the umbrella array may not be designed to fit within a cavity. Additionally, or alternatively, the uniform potential distribution of an umbrella array, as a result of the electrodes being jointly connected to one another, results in a tissue ablation geometry that is not adjustable without physically moving the umbrella array, thus resulting in similar problems as long rigid needle-based RF applicators.

The system of the present disclosure can be used during an ablation procedure to monitor ablation progress of a given tissue, which may include obtaining measurements related to physiological parameters, including tissue impedance, temperature, and the like. The system is configured to actively sense that status of the tissue, which may include an accurate estimation of the state of the tissue to be ablated, currently undergoing ablation, or having undergone ablation. Furthermore, the system is configured to sense the ablation depth in relevant tissue types as a result of the measurements. In response, the system can control energy emission from the ablation device based on the sensed tissue status and ablation depth, specifically adjusting the energy emission so as to adapt to the unique heterogeneity and different tissue densities, which may be commonly found in breast tissue, for example. Thus, the system of the present disclosure allows for real-, or near-real-, time ablation optimization for any patient regardless of varying tissue composition and density (i.e., varying breast density from patient to patient) by accounting for individual tissue density and conductivity, and further allows for the active avoidance of vital organs and any critical internal/external structures (e.g., bone, muscle, skin) in close proximity to the targeted ablation site.

The system of the present disclosure generally includes a controller configured to control energy emission from an electrode array of an ablation device based, at least in part, on ablation feedback received during an ablation procedure with the ablation device. The controller is configured to receive feedback data associated with at least one of operation of the electrode array and any tissue adjacent to the electrode array during an ablation procedure. The feedback data includes one or more measurements, including, but not limited to, an elapsed time during an ablation period, electrical conductivity or complex impedance (including impedance magnitude and phase) associated with one or more conductive wires of the electrode array, electrical current supplied to the one or more conductive wires, temperature of tissue adjacent to the electrode array, photonic properties of the tissue adjacent to the electrode array, and a combination thereof.

The controller is further configured to generate an ablation pattern for controlling energy emission from the electrode array of the ablation device in response to the received feedback data. The ablation pattern may include, but is not limited to, a selected one or more conductive wires from a plurality of conductive wires of the electrode array, to receive electrical current for energy emission, a level of electrical current to be supplied to a selected one or more conductive wires, a length of elapsed time during which electrical current is to be supplied to a selected one or more conductive wires, one or more intervals over which electrical current is to be supplied to a selected one or more conductive wires, and a combination thereof. The electrode array of the ablation device may include independent conductive wires configured to independently receive electrical current and may be arranged around sides or faces of a distal probing end of the ablation device. Therefore, in some embodiments, the ablation pattern may include a selected one, or a selected set of two or more, of the plurality of conductive wires resulting in emission of energy therefrom corresponding to a portion of the electrode array, and thus a side or face of the distal probing end of the device, thereby resulting in targeted ablation of adjacent tissue.

The generation of the ablation pattern may include processing the feedback data in real-, or near-real-, time and generating ablation status mapping based on the processed feedback data. The ablation status mapping provides an estimation of the state of the tissue to be ablated, currently undergoing ablation, or having undergone ablation. The generation of the ablation status mapping may include processing of the feedback data in accordance with at least the formula: (t, s, init_local_Z[], init_global_Z[], current_local_Z[], current_global_Z[], x, y, z) AblationStatus, wherein ‘t’ indicates an elapsed time in seconds, ‘s’ indicates a size of an ablating end of the ablation device, ‘Z’ indicates impedance, ‘[]’ indicates arrays with length of a number of conductive wires, and ‘x,y,z’ are coordinates of a sub volume of tissue.

The generation of the ablation pattern may further include a combination of ablation status mapping data with an electrode activation algorithm for assignment of one or more ablation control parameters for selective conductive wire activation for subsequent targeted ablation of adjacent tissue. Accordingly, the system may include an ablation mapping module and an ablation geometry shaping module, the ablation mapping module configured to receive and process the feedback data and transmit mapping data to the ablation geometry shaping module configured to process the mapping data to generate the ablation pattern. The ablation geometry shaping module may be configured to transmit the ablation pattern to an electrode connection multiplexer controller, which is configured to supply electrical current to a selected one, or set of two or more, conductive wires in response to the ablation pattern.

The systems and methods of the present disclosure can help to ensure that all microscopic disease in the local environment has been treated. This is especially true in the treatment of tumors that have a tendency to recur. Furthermore, by providing custom ablation shaping, based, at least in part, on feedback received during an ablation procedure (e.g., measured physiological parameters, including tissue impedance, temperature, and the like), the system can control a single ablation device to provide numerous RF energy emission shapes or profiles tailored to the properties (e.g., conductivity, depth, etc.) for any given target tissue. Accordingly, the system of the present invention is capable of providing optimal transmission of energy and subsequent uniform heating throughout a heterogeneous tissue during the ablation procedure. Additionally, by providing numerous RF energy emission shapes or profiles from a single ablation device, the system allows for non-uniform ablation to occur. This is particularly useful in controlling ablation shape so as to avoid vital organs and any critical internal/external structures (e.g., bone, muscle, skin) in close proximity to the tumor site, while ensuring that residual marginal tissue within the local environment has been treated.

It should be noted the devices of the present disclosure are not limited to such post-surgical treatments and, as used herein, the phrase “body cavity” may include non-surgically created cavities, such as natural body cavities and passages, such as the ureter (e.g. for prostate treatment), the uterus (e.g. for uterine ablation or fibroid treatment), fallopian tubes (e.g. for sterilization), and the like. Additionally, or alternatively, tissue ablation devices of the present disclosure may be used for the ablation of marginal tissue in various parts of the body and organs (e.g., lungs, liver, pancreas, etc.) and is not limited to treatment of breast cancer.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIG. 1 is a schematic illustration of an ablation system consistent with the present disclosure;

FIG. 2 is a block diagram illustrating an ablation monitoring and shaping system of a device controller of the system of FIG. 1 in greater detail;

FIG. 3 is a block diagram illustrating the flow of RF current during an ablation procedure using the ablation monitoring and shaping system consistent with the present disclosure;

FIG. 4 is a block diagram illustrating the flow of ablation feedback data using the ablation monitoring and shaping system consistent with the present disclosure

FIG. 5 is a simplified block diagram illustrating the ablation monitoring and shaping system consistent with the present disclosure:

FIGS. 6A and 6B are perspective side views of a distal probing end of an exemplary ablation device compatible with the system of the present disclosure, illustrating different faces of the distal probing end;

FIGS. 7A and 7B are plots of measured complex impedance values (impedance magnitude) relative to a ablation depth from one face of the distal probing end of the ablation device using device-only electrodes (FIG. 7A) and external electrodes (FIG. 7B);

FIGS. 8A and 8B are plots of measured complex impedance values (phase) relative to a ablation depth from one face of the distal probing end of the ablation device using device-only electrodes (FIG. 8A) and external electrodes (FIG. 8B);

FIG. 9 is a plot of architecture of a neural network used by the ablation mapping subsystem for providing ablation status;

FIG. 10 is a diagram illustrating the timing of ablation, measurement, and computation of the device consisting with the present disclosure.

FIG. 11 is an illustration of a subvolume of target tissue relative to an activated face of the distal probing end of the ablation device;

FIG. 12 is an exemplary output of ablation status mapping converted to ablation depth based on feedback data collected during an ablation procedure comprising electrode activation in a programmed activation algorithm;

FIG. 13 is a schematic illustration of a multiplexer/switch (Mux) for use with the ablation monitoring and shaping system consistent with the present disclosure;

FIG. 14 is a schematic illustration of exemplary matrix switch circuitry for use with the ablation monitoring and shaping system consistent with the present disclosure;

FIGS. 15 and 16 are schematic illustrations of hardware setups for different lines of multiplexers/switches (Muxes) for connecting two or more electrodes associated with different faces of the ablation device;

FIG. 17 is a schematic illustration of the matrix switch circuitry of FIG. 15 for connecting lines on the ablation device;

FIGS. 18A and 18B illustrate cutting methods of tissue having undergone targeted ablation using an ablation device controlled with the ablation monitoring and shaping system of the present disclosure. The cutting methods include splitting of a block of tissue into distinct portions to provide views of tissue corresponding to faces of the ablation device;

FIGS. 19A and 19B are images of a first tissue sample split into distinct portions in accordance with the cutting methods illustrated in FIGS. 18A and 18B, the images showing controlled ablation of the tissue in a targeted manner according to a first ablation pattern/geometry; and

FIGS. 20A and 20B are images of a second tissue sample split into distinct portions in accordance with the cutting methods illustrated in FIGS. 18A and 18B, the images showing controlled ablation of the tissue in a targeted manner according to a second ablation pattern/geometry.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present disclosure is generally directed to a system for monitoring and controlling an ablation device to cause the ablation device to emit energy in an optimal shape or pattern so as to deliver treatment for the ablation and destruction of a targeted portion of marginal tissue around the tissue cavity.

In particular, the system of the present disclosure can be used during an ablation procedure to monitor ablation progress of a given tissue, which may include obtaining measurements related to physiological parameters, including tissue impedance, temperature, and the like. The system is configured to actively sense that status of the tissue, which may include an accurate estimation of the state of the tissue to be ablated, currently undergoing ablation, or having undergone ablation. Furthermore, the system is configured to sense the ablation depth in relevant tissue types as a result of the measurements. In response, the system can control energy emission from the ablation device based on the sensed tissue status and ablation depth, specifically adjusting the energy emission so as to adapt to the unique heterogeneity and different tissue densities, which may be commonly found in breast tissue, for example. Thus, the system of the present disclosure allows for real-, or near-real-, time ablation optimization for any patient regardless of varying tissue composition and density (i.e., varying breast density from patient to patient) by accounting for individual tissue density and conductivity, and further allows for the active avoidance of vital organs and any critical internal/external structures (e.g., bone, muscle, skin) in close proximity to the targeted ablation site.

The system of the present disclosure generally includes a controller configured to control energy emission from an electrode array of an ablation device based, at least in part, on ablation feedback received during an ablation procedure with the ablation device. The controller is configured to receive feedback data associated with at least one of operation of the electrode array and any tissue adjacent to the electrode array during an ablation procedure. The feedback data includes one or more measurements, including, but not limited to, an elapsed time during an ablation period, electrical conductivity or complex impedance (including impedance magnitude and phase) associated with one or more conductive wires of the electrode array, electrical current supplied to the one or more conductive wires, temperature of tissue adjacent to the electrode array, photonic properties of the tissue adjacent to the electrode array, and a combination thereof.

The controller is further configured to generate an ablation pattern for controlling energy emission from the electrode array of the ablation device in response to the received feedback data. The ablation pattern may include, but is not limited to, a selected one or more conductive wires from a plurality of conductive wires of the electrode array, to receive electrical current for energy emission, a level of electrical current to be supplied to a selected one or more conductive wires, a length of elapsed time during which electrical current is to be supplied to a selected one or more conductive wires, one or more intervals over which electrical current is to be supplied to a selected one or more conductive wires, and a combination thereof. The electrode array of the ablation device may include independent conductive wires configured to independently receive electrical current and may be arranged around sides or faces of a distal probing end of the ablation device. Therefore, in some embodiments, the ablation pattern may include a selected one, or a selected set of two or more, of the plurality of conductive wires resulting in emission of energy therefrom corresponding to a portion of the electrode array, and thus a side or face of the distal probing end of the device, thereby resulting in targeted ablation of adjacent tissue.

The systems and methods of the present disclosure can help to ensure that all microscopic disease in the local environment has been treated. This is especially true in the treatment of tumors that have a tendency to recur. Furthermore, by providing custom ablation shaping, based, at least in part, on feedback received during an ablation procedure (e.g., measured physiological parameters, including tissue impedance, temperature, and the like), the system can control a single ablation device to provide numerous RF energy emission shapes or profiles tailored to the properties (e.g., conductivity, depth, etc.) for any given target tissue. Accordingly, the system of the present invention is capable of providing optimal transmission of energy and subsequent uniform heating throughout a heterogeneous tissue during the ablation procedure. Additionally, by providing numerous RF energy emission shapes or profiles from a single ablation device, the system allows for non-uniform ablation to occur. This is particularly useful in controlling ablation shape so as to avoid vital organs and any critical internal/external structures (e.g., bone, muscle, skin) in close proximity to the tumor site, while ensuring that residual marginal tissue within the local environment has been treated.

It should be noted the devices of the present disclosure are not limited to such post-surgical treatments and, as used herein, the phrase “body cavity” may include non-surgically created cavities, such as natural body cavities and passages, such as the ureter (e.g. for prostate treatment), the uterus (e.g. for uterine ablation or fibroid treatment), fallopian tubes (e.g. for sterilization), and the like.

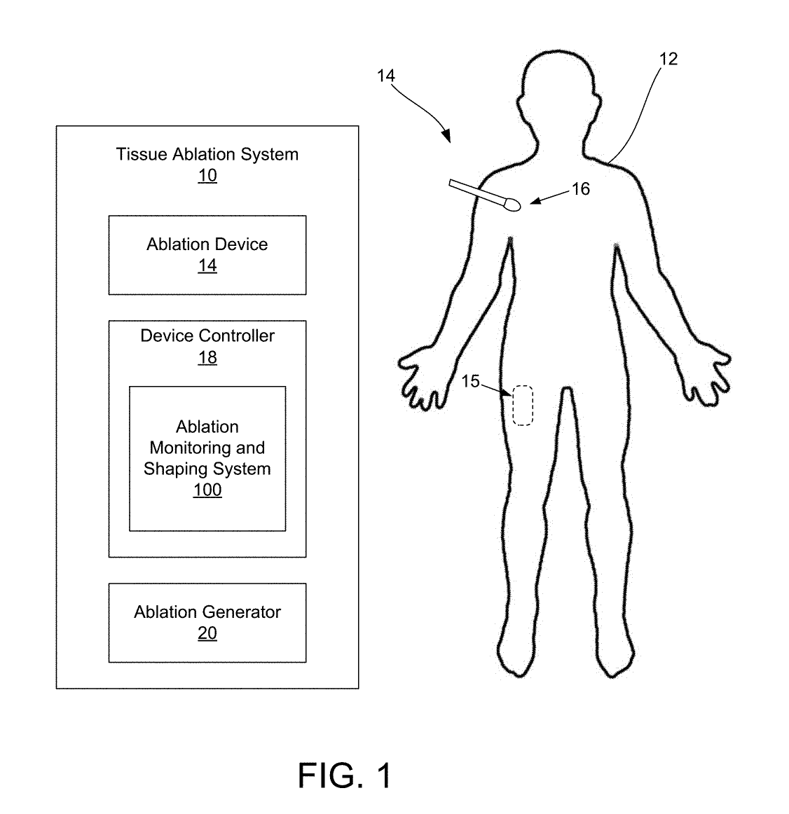

FIG. 1 is a schematic illustration of an ablation system 10 for providing targeted ablation of marginal tissue during a tumor removal procedure in a patient 12. The ablation system 10 generally includes an ablation device 14, which includes a probe having a distal probing tip 16. The ablation device 14 may be coupled to a device controller 18 and an ablation generator 20 over an electrical connection. In some embodiments, the ablation device may also be coupled to an irrigation pump or drip over a fluid connection (for providing irrigation or other fluid flow before, during, or after an ablation procedure).

As will be described in greater detail herein, the device controller 18 includes an ablation monitoring and shaping system 100 configured to provide monitoring and collection of data during an ablation procedure and to further provide custom ablation shaping, which includes the creation of either user-defined ablation patterns or geometries from the ablation device 14 or automatically generated ablation patterns or geometries that are based, at least in part, on the collected ablation data. In some cases, the device controller 18 may be housed within the ablation device 14. In some embodiments, the ablation generator 20 may also connected to a return electrode 15 that is attached to the skin of the patient 12. However, it should be noted that the ablation device 14 includes bipolar operating mode, and thus, in some embodiments, the use of the separate return electrode 15 is unnecessary.

As will be described in greater detail herein, during an ablation treatment, the ablation generator 20 may generally provide RF energy (e.g., electrical energy in the radiofrequency (RF) range (e.g., 350-800 kHz)) to an electrode array of the ablation device 14, as controlled by the device controller 18. The RF energy travels through the blood and tissue of the patient 12 and, in the process, ablates the region(s) of tissues adjacent to portions of the electrode array that have been activated.

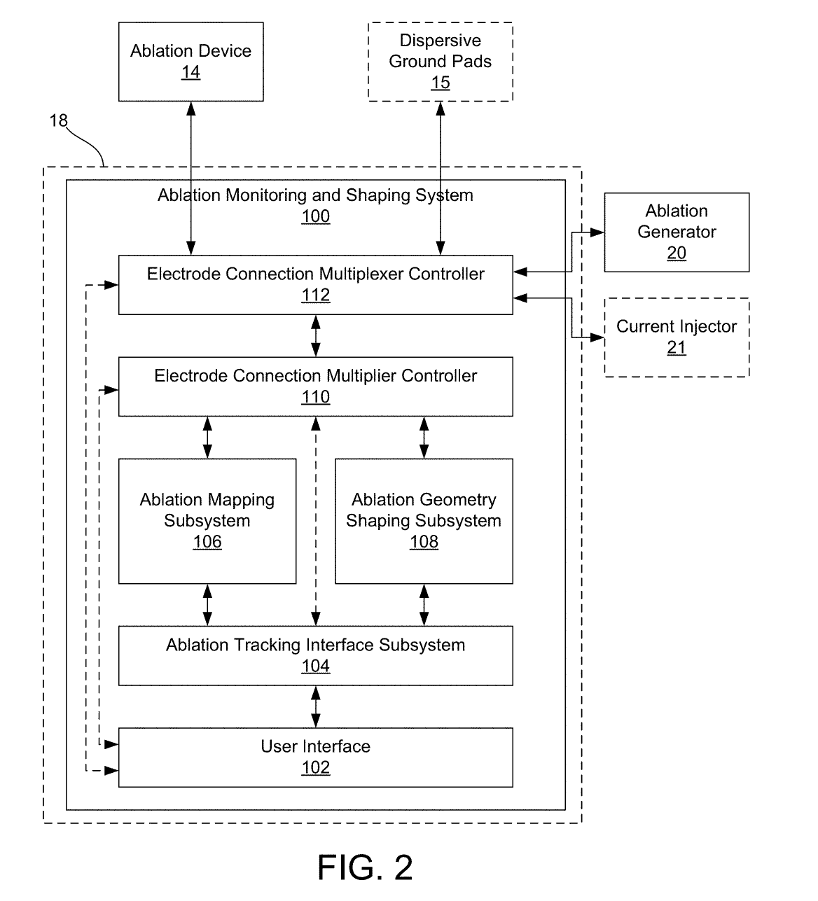

FIG. 2 is a block diagram illustrating the ablation monitoring and shaping system 100 of the device controller 18. The ablation monitoring and shaping system 100 is configured to generate an ablation pattern for controlling energy emission from an electrode array provided on the ablation device 14 in response to feedback data collected by the system 100 during an ablation procedure.

It should be noted that the ablation device 14 may include an electrode array including at least two or more electrodes, which may be in the form of conductive wires, that are able to be independently controlled (i.e., indepedently received electrical current and be activated independent of one another). Such a design allows for the system 100 to independently control activation of each conductive wire (e.g., control the delivery of energy in the form of electrical current from the ablation generator 20 and subsequent emission of RF energy in response thereto). The device controller 18 is configured to selectively control the supply of electrical current to each of the conductive wires via the system 100. It should further be noted that the at least two or more electrodes of the electrode array may be positioned on different portions of the distal probing tip 16 of the ablation device 14, such as different sides or faces. Accordingly, upon activation of one of the electrodes, RF energy is emitted from the corresponding side or face of the distal probing tip 16 that is associated with the activated electrode.

Accordingly, the ablation pattern provided by the system 100 may include, but is not limited to, a selected one or more conductive wires from a plurality of conductive wires of the electrode array, to receive electrical current for energy emission, a level of electrical current to be supplied to a selected one or more conductive wires, a length of elapsed time during which electrical current is to be supplied to a selected one or more conductive wires, one or more intervals over which electrical current is to be supplied to a selected one or more conductive wires, and a combination thereof. Therefore, in some embodiments, the ablation pattern may include a selected one, or a selected set of two or more, of the plurality of conductive wires resulting in emission of energy therefrom corresponding to a portion of the electrode array, and thus a side or face of the distal probing end 16 of the device 14, thereby resulting in targeted ablation of adjacent tissue.

The ablation monitoring and shaping system 100 includes one or more of the following: a user interface 102; an ablation tracking interface subsystem 104; an ablation mapping subsystem 106; an ablation geometry shaping subsystem 108; an electrode connection multiplier controller 110; and an electrode connection multiplexer controller 112. It should be noted that the dashed connections (between the user interface 102 and electrode connection multiplier controller 110 and the electrode connection multiplexer controller 112) indicate fail-safes and out-of-band control lines not used, or intended for use, during normal operation. However, in the event that one or more of the components fail to operate as intended, the user may override such components so as to directly control activation of one or more conductive wires 28.

As previously described, the specific design of the electrode array (e.g., plurality of conductive wires electrically isolated and independent from one another) allows for each conductive wire to receive energy in the form of electrical current from the ablation generator 20 and emit RF energy in response. In particular, the device controller 18 allows for individual conductive wires, or a designated combination of conductive wires, to be controlled so as to result in the activation (e.g., emission of RF energy) of corresponding portions of the electrode array.

In some embodiments, the device controller 18, specifically by way of the ablation monitoring and shaping system 100, provides a user with the ability to manually control the supply of electrical current to each of the conductive wires. More specifically, the user interface 102 may provide a user with the ability to create custom ablation shapes or patterns, or further manipulate ablation parameters (e.g., timing and intensity) via an interactive interface, which may be in the form of a graphical user interface (GUI) provided on a display of the device controller 18. Accordingly, the ablation monitoring and shaping system 100 may allow a user to manually control emission from the electrode array and customize the ablation shape or geometry as they see fit.

In other embodiments, the ablation monitoring and shaping system 100 may be configured to automatically provide custom ablation shaping in addition, or alternatively, to manual input from a user. For example, the device controller 18 may be configured to provide ablation status mapping based on real-time data collection (e.g., temperature and conductivity measurements (complex impedance measurements) from one or more of the conductive wires) so as to provide an estimation of the state of the tissue during an RF ablation procedure. In particular, the system 100 can be used during an ablation procedure to monitor ablation progress of a given tissue, which may include obtaining measurements related to physiological parameters, including tissue impedance, temperature, and the like. The system 100 is configured to actively sense that status of the tissue, which may include an accurate estimation of the state of the tissue to be ablated, currently undergoing ablation, or having undergone ablation. Furthermore, the system 100 is configured to sense the ablation depth in relevant tissue types as a result of the measurements. In response, the system 100 can control energy emission from the ablation device based on the sensed tissue status and ablation depth, specifically adjusting the energy emission so as to adapt to the unique heterogeneity and different tissue densities, which may be commonly found in breast tissue, for example. In some embodiments, the system 100 is configured to generate ablation status mapping of a target tissue based, at least in part, on characterizing temporal changes in conductivity of a target tissue during ablation and correlating such changes with temperature and cell viability. The ablation status mapping may then be combined with an electrode activation algorithm for the assignment of parameters for selective electrode activation for ablation shaping.

Accordingly, the automatic custom ablation shaping feature of the present invention allows for spatial resolution of the ablation mapping and shaping systems to occur in vitro and further determine the depths from the electrode which the mapping/sensing system can provide reliable estimations. Thus, the system 100 allows for real-, or near-real-, time ablation optimization for any patient regardless of varying tissue composition and density (i.e., varying breast density from patient to patient) by accounting for individual tissue density and conductivity, and further allows for the active avoidance of vital organs and any critical internal/external structures (e.g., bone, muscle, skin) in close proximity to the targeted ablation site.

In order to achieve the capability of ablation status mapping, the ablation monitoring and shaping system 100 is configured to collect data for a machine learning model and then use the model to map ablation status in real time. The data collected includes, but is not limited to, temperature measurements, conductivity or complex impedance measurements, and photonic properties of the target tissue. By measuring time and the change in impedance (real or complex), temperature, and/or photonic properties of the target tissue, the system 100 is configured to determine the ablation shape or geometry (energy emission from electrode array) in real-, or near-real-, time.

Since each conductive wire in the electrode array of the device 14 is electrically independent, each conductive wire can be connected in a fashion that allows for impedance measurements using bipolar impedance measurement circuits. For example, the conductive wires can be configured in such a fashion that tetrapolar or guarded tetrapolar electrode configurations can be used. For instance, one pair of conductive wires could function as the current driver and the current return, while another pair of conductive wires could function as a voltage measurement pair. The dispersive ground pads 15 can also function as current return and voltage references. Their placement dictate the current paths and thus having multiple references can also benefit by providing additional paths for determining the ablation status of the tissue.

The electrode connection multiplexer controller 112 is configured to collect the data in the form of local impedances (impedances between conductive wires on the distal tip) and global impedances (impedances between conductive wires and global dispersive return 15) and further transmit such data to the ablation mapping subsystem 106. A Kelvin electrode configuration driven with 500 μA at 200 kHz (for filtering from the 470 kHz RF signal) may be used in order to measure these impedances.

The ablation mapping subsystem 106 is configured to analyze the impedance data with time elapsed in order to form a judgment of the ablation status of certain parts of the entire ablation volume. In particular, the ablation mapping subsystem 106 may include custom, proprietary, known and/or after-developed analysis code (or instruction sets), hardware, and/or firmware that are generally well-defined and operable to receive one or more sets of data and estimate an ablation status of local target tissue sub volumes based on analysis of such data. Thus, the ablation mapping subsystem 106 may utilize a specific input model in order to output an ablation status integer for any sub volume of the ablation volume. The input model is as follows:

(t, s, init_local_Z[], init_global_Z[], current_local_Z[], current_global_Z[], x, y, z)→AblationStatus

where ‘t’ indicates time in seconds, ‘s’ indicates the size of the applicator (diameter, area, volume, etc. of the distal tip), ‘Z’ indicates impedance, ‘[]’ indicates arrays with length of the number of conductive wires, and ‘x,y,z’ are the coordinates of the sub volume.

As in the input model provided above, each sub volume the ablation map may include five possible statuses: “0” indicating no ablation occurring, “1” indicating that heating is occurring, “2” indicating that instantaneous ablation or coagulation has begun (the tissue has reached a temperature of 60° C.), “3” indicating that ablation has occurred, and “4” indicating that desiccation (vaporization) is occurring. In order to develop the classification model, benchtop ablations are performed where the following training data is collected: time, init_local_Z[], init_global_Z[], current_local Z[], current_global Z[], and for a set of radii (0.25, 0.5, 0.75, 1.0, 1.25, 1.5 cm) surrounding the applicator, the exact temperature, which translates to the ablation status (0 for initial temperature, 1 for ≥40° C., 2 for ≥50° C., 3 for ≥60° C., 4 for ≥100° C.). This method of ablation mapping is also designed to be mostly heterogeneity-invariant, since local impedances are inputs into the model, which treat the heterogeneous tissues as different tissue types present.

In order to obtain reference tissue ablation parameters, the training data may then be input into multiple supervised machine learning algorithms, where the most accurate classifier will be used for the real-time system. Training data may be collected within ex vivo bovine and porcine liver blocks of 10 cm by 10 cm by 10 cm. The tissue can be placed in a saline bath such that the global ground is simulated as far-field to prevent optimistic global impedance measurements. Verifications on the classifier will be performed after the model is learned to ensure success criteria, including controls with and without RF energy applied.

The target endpoint is 90% accuracy (with zero false ablated statuses) of ablation status mapping with 1.0 mm of spatial sub volume resolution for the local field (≤1.0 cm depth from applicator surface). Additional success criteria may include the accuracy of ablation status mapping up to 3.0 mm sub volume resolution into the sub global field (1.0-2.0 cm depth from applicator surface).

If the classifier fails to classify based only on initial and changes in impedance, then an additional parameter, the estimated local tissue conductivity, will be added to the model. The estimated conductivity is covered within the model by the initial and early-time impedances, but a more explicit variable may be required. If the target endpoint spatial resolutions failed to be realized, then the electrodes will be increased in number to increase density for higher spatial resolution.

The ablation geometry shaping subsystem 108 is configured to receive output data from the ablation mapping subsystem 106, specifically ablation status mapping data via the ablation tracking interface subsystem 104, and determine a specific ablation shape or geometry to output (e.g., identify specific conductive wires or combination of conductive wires to apply power to and the specific parameters) in order to achieve the desired custom ablation shape based on the ablation status mapping. In particular, the ablation geometry shaping subsystem 108 may rely on an electrode activation algorithm necessary to operate the network of solid-state relays (also known as a crossbar) that connect the conductive wires to the radiofrequency power generator 20. The ablation geometry shaping subsystem 108 may generate ablation shape data based on processing of the ablation status mapping data via the electrode activation algorithm.

The ablation geometry shaping subsystem 108 may then transmit ablation shape data to the electrode connection multiplexer controller 112 for activation of specific conductive wires, or combinations of conductive wires, so as to achieve the desired ablation shape. For example, the electrode connection multiplier controller 110 may be configured to physically operate solid-state relays on the electrode connection multiplexer (the electrode-switching/power-switching circuit), connecting the electrodes needed to RF power. By time-division multiplexing, different conductive wires in a manner similar to pulse width modulation (PWM), where the conductive wires are connected to power for a specified duration and then disconnected in a repeated pattern. Time-multiplexing may be especially important for deeper ablations that are geometrically between multiple conductive wires, in which the theoretical circuit relies on heat transfer to nearby (i.e., not currently electrically-conducting) tissues and only the concentration of heat in the desired zone due to the combined efforts of the conductive wires activating in the multiplexed fashion.

The ablation mapping subsystem 106 and ablation geometry shaping system 108 may be configured to continuously operate during a procedure so as to provide up-to-date information which may further improve the accuracy and safety of the ablation procedure. For example, ablation status mapping data may be continuously generated and fed into the ablation geometry shaping system 108 so as to continuously generate ablation shaping data, which may be used to either validate the current ablation energy applied, or to update or correct the ablation shape (i.e., indicate where to continue ablation or when to stop ablation). It should further be noted that ablation mapping status can be displayed to a user using a 3D visualization, which can be controlled by the user interface 102 (e.g., touchscreen or the like) similar to a 3D map application. Each layer of tissue may be displayed as being somewhat transparent so as to allow for the operator to see which regions are ablated and which are not.

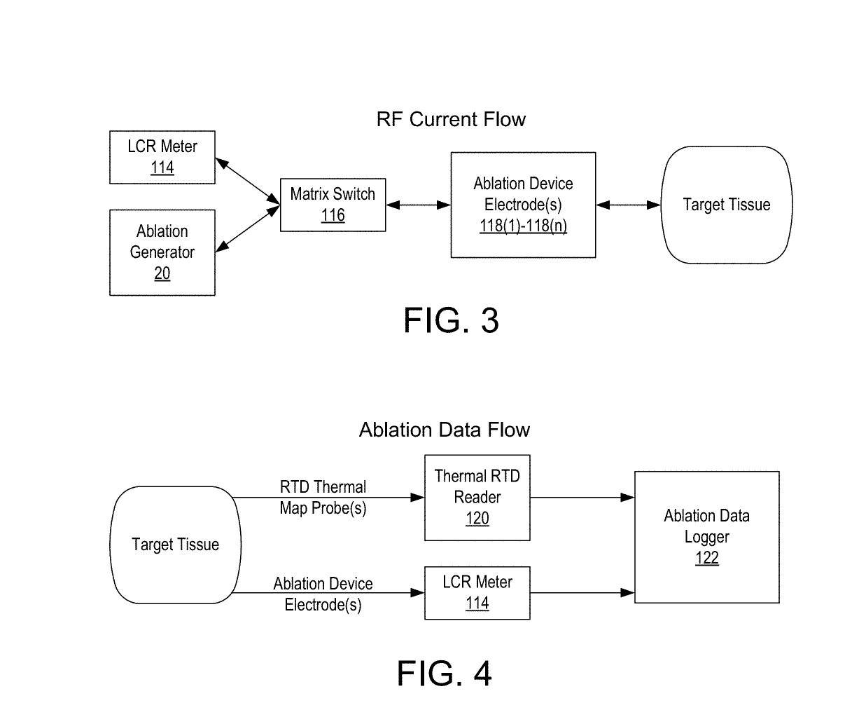

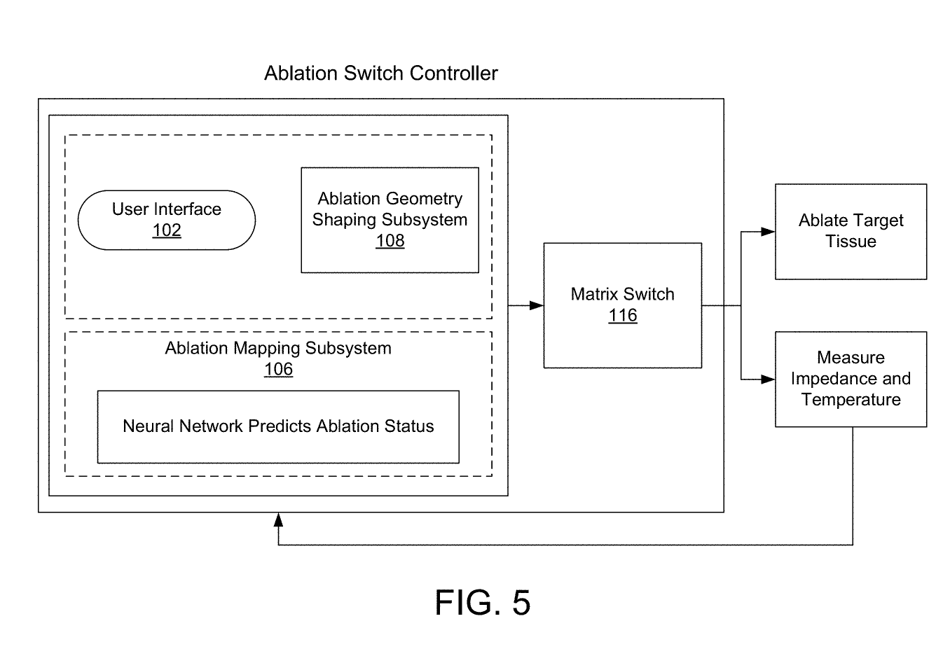

The process of collecting feedback data and subsequent generating ablation patterns or geometries by the system 100 generally includes three separate flows: an RF current flow; ablation feedback data flow; and a control data flow. FIGS. 3 and 4 are block diagrams illustrating the flow of RF current during an ablation procedure and the subsequent flow of ablation feedback data to the system 100. FIG. 5 is a simplified block diagram illustrating the ablation monitoring and shaping system 100 providing subsequent control data flow back to the device 14 to control emission therefrom.

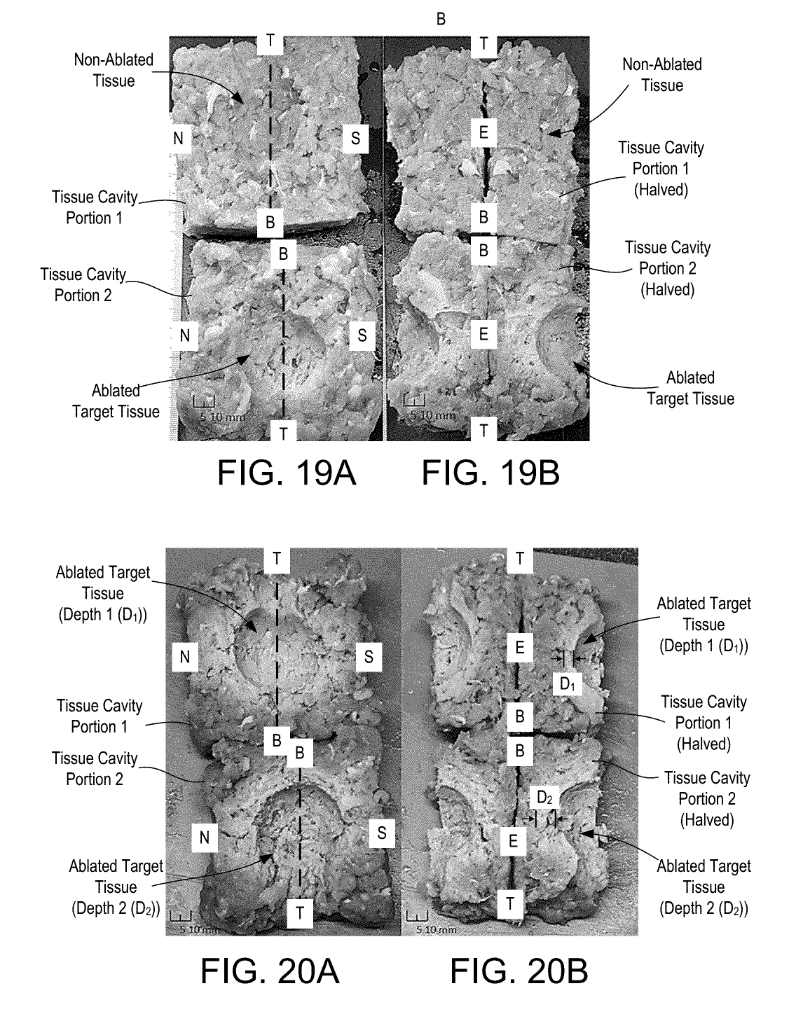

During testing of the system 100, the electrodes of the device 14 were connected to a crossbar matrix switch 116, which may be visualized as a grid (shown in FIGS. 16 and 19), in which any input can be connected to any output and vice versa. The use of a crossbar matrix switch 116 allows for the individual electrodes of the device 14 to be switched between complex impedance measurement of an RF current, as measured by an LCR meter 114 and RF ablation current, as supplied by the ablation generator 20. The LCR meter 114 is generally a type of electronic test equipment used to measure the inductance (L), capacitance (C), and resistance (R) of an electronic component.

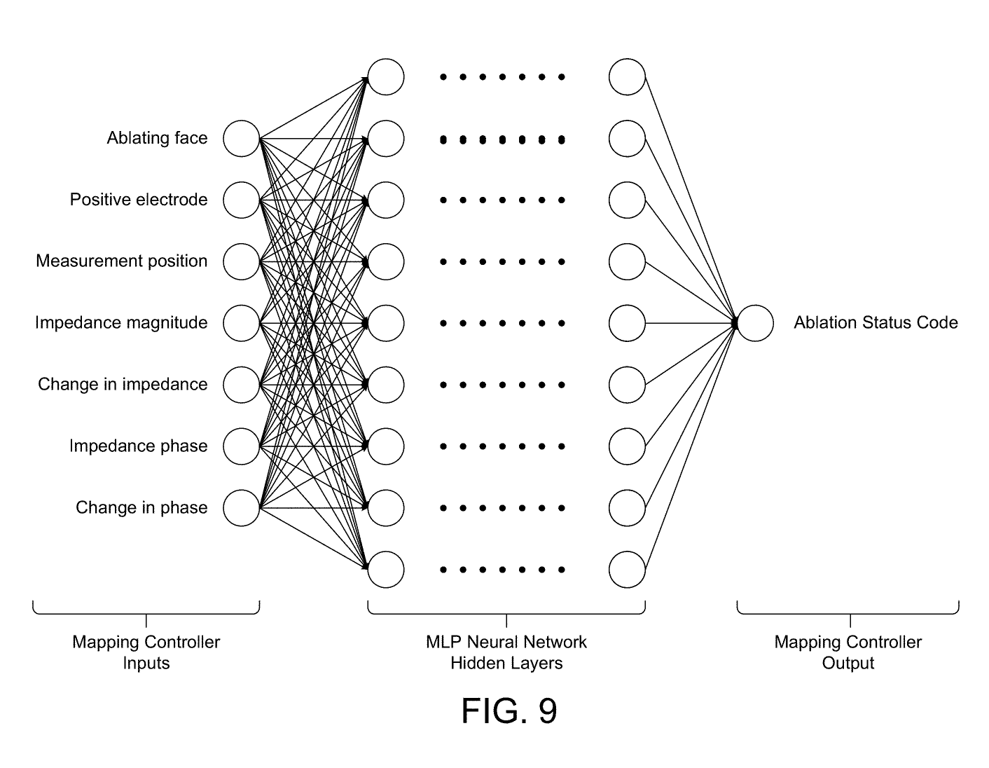

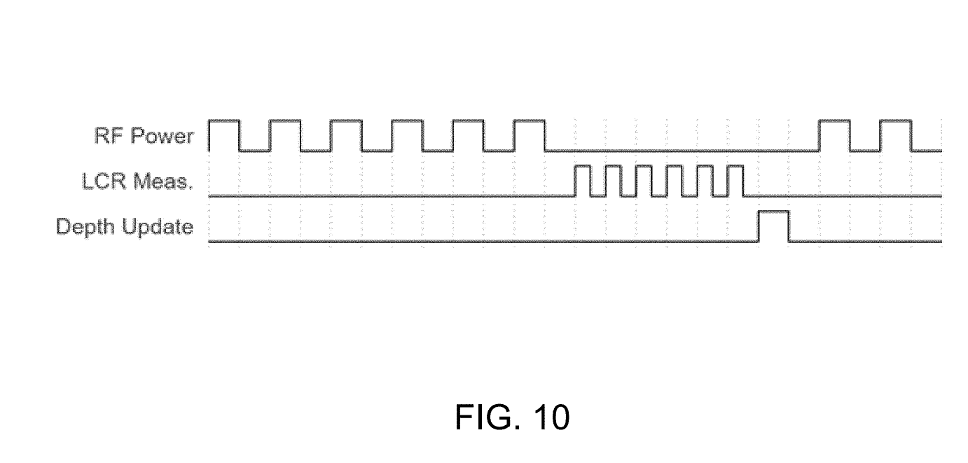

The RF current flow to the target tissue begins with electrical current generated either by the LCR meter 114 or the ablation generator 20. The current flows through the matrix switch 116, which allows only one RF current source to be connected at a time, and into target tissue via the electrodes 118 on the device 14 that are connected by the matrix switch 116. Similarly, the matrix switch 116 also allows for connection with external electrodes. However, as will be described in greater detail herein (FIGS. 9A-9B and 10A-10B), experimental data shows that external electrodes were not required for the millimeter-resolution estimation objective.

The ablation data flow of FIG. 4 is the flow of ablation feedback training data collected by temperature probes and the LCR meter 114 to data logs (ablation data logger 112) for machine learning processing. For example, thermal data is recorded by a signal-conditioning RTD analog-to-digital converter 120. The complex impedance data is measured and recorded by the LCR meter 114. Both data streams are recorded and processed by a processor and/or additional hardware provided in the system 100.

The ablation switch controller components illustrated in FIG. 5, specifically the ablation mapping subsystem 106 and ablation shaping subsystem 108 are ultimately capable of controlling RF emission from the device 14 based on the collected feedback data. The primary device controlled by the logic executed by either of the ablation mapping subsystem 106 and ablation shaping subsystem 108 is the matrix switch 116, which, in turn, is configured to select either ablation or measurement modes for the system 100. The human-machine interface (user interface 102), in which the user may select the custom ablation geometry desired in the target tissue via the graphical user interface, may be part of the starting node of the ablation control flow illustrated in FIG. 5. To close the control loop, the system 100 sends the measured complex impedance data and thermal data to the mapping controller, which, to the user, displays the ablation depth of the target tissue present at a given face or side of the distal probing tip 16 (corresponding to the associated electrode), and, based on logic, is configured to adjust the ablation procedure accordingly.

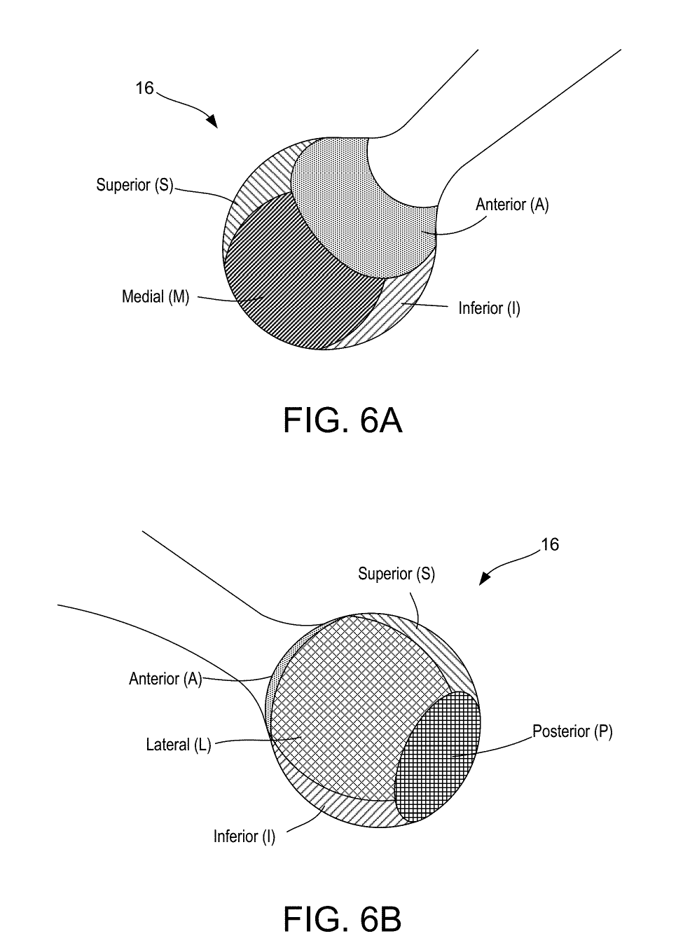

FIGS. 6A and 6B are perspective side views of a distal probing end 16 of an exemplary ablation device 14 compatible with the system 100 of the present disclosure. As previously described, the ablation device 14 may include a plurality of electrodes, each of which may be positioned on a corresponding portion of the distal probing tip 16. Accordingly, upon independent activation of one of the electrodes, RF energy is emitted from the corresponding portion of the distal probing tip 16. For example, as shown in FIGS. 6A and 6B, the distal tip 16 may include six sides or faces, which may include superior (S), inferior (I), lateral (L), medial (M), anterior (A), and posterior (P) faces. In this embodiment, the tip 16 may be placed in a cavity (i.e., lumpectomy cavity), such that each of the faces touches a clinical margin. Individual control of the electrodes and thus control over RF emission from the corresponding faces is of particular importance. In many procedures involving ablation of margin in a cavity, the A face is likely the closest face to the patient's skin whereas the P face is likely the closest to the chest wall. Thus, control over energy emission from the anterior (A) and posterior (P) faces may be of particular importance due to their increased probability of being positioned near critical structures during ablation of marginal tissue within a cavity. The system 100 allows for individual control of margin sides to achieve the desired ablation geometry and more importantly, to avoid ablating or damaging these critical structures. As described in greater detail herein, data collected during testing illustrates that the system 100 was able to control energy emission from each of the faces of the device 14 to such a degree that energy was able to be delivered differentially within the target tissue to address heterogeneity and that damage to critical structures, such as skin and muscle was able to be avoided. For example, the system 100 is able to control title to no ablation in either of the A face or P face occurs, while desired ablation in other faces was achieved, which is clinically important to prevent skin damage.

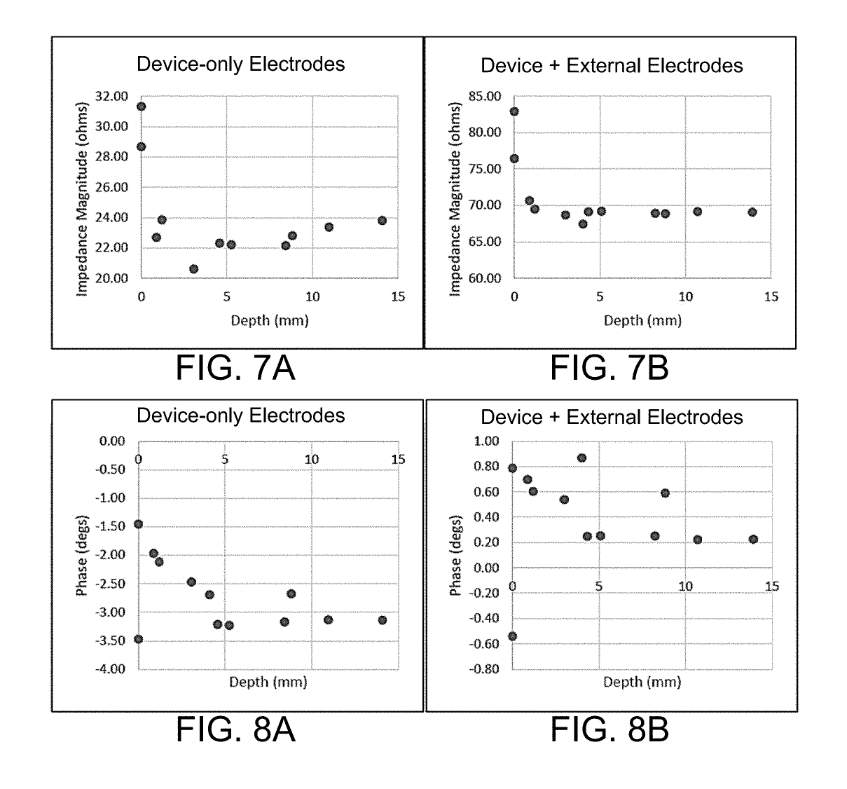

FIGS. 7A and 7B are plots of measured complex impedance values (impedance magnitude) relative to a ablation depth from one face of the distal probing end of the ablation device using device-only electrodes (FIG. 7A) and external electrodes (FIG. 7B). FIGS. 8A and 8B are plots of measured complex impedance values (phase) relative to a ablation depth from one face of the distal probing end of the ablation device using device-only electrodes (FIG. 8A) and external electrodes (FIG. 8B).

Complex electrical impedance (magnitude and phase) between electrode sets was collected at 100 kHz (the frequency range of RF ablation) during ablation of the three tissue different models. The electrode sets included on-device electrodes and external electrodes. Similar morphologies of the individual components of the complex impedance data (impedance magnitude and phase) comparing the data collected using only on-device electrodes versus data collected using both on-device and external electrodes showed that using external electrodes were not required for pattern recognition. Using external electrodes guarantees that the measured current paths pass through all tissues of the target tissue and allows for electro-sense using the on-device electrodes. However, given that the depth relationship between device-only and external electrodes was not significantly different in the tissue models studied within this proof-of-concept, the external electrodes were removed from the final system for simplicity of user interviews and deployment.

The distinct morphology of the change in impedance over depth of ablation of one side of the target tissue margin (as shown in FIGS. 7A and 7B) illustrates that, while non-linear, a clear relationship between electrical impedance and ablation depth is present. This relationship is in line with the physical properties of biological tissue, which presents different values of electrical conductivity (reciprocal of per-volume impedance magnitude, or electrical volume resistivity) based on the temperature of the volume. The electrical conductivity of biological tissue increases with temperature until protein coagulation and vaporization, which causes the electrical conductivity of the tissue to plummet as the electrolyte that carries RF current leaves the tissue volume.

Since RF ablation is based on heat generation, the relationship between depth and decreasing electrical impedance magnitude values aligns with the physical theory, where electrical conductivity of tissue subvolumes increase as more subvolumes of tissue increase in temperature, decreasing overall impedance magnitude. The impedance phase (shown in FIGS. 8A and 8B) affords another measure for the ablation depth relationship, where a capacitance is formed, likely due to protein coagulation and RF current application by the electrodes causing ion migration and thus creating a virtual capacitor of the target tissue.

These two direct relationships allow for machine learning algorithms to be used to classify and estimate the ablation geometry map without additional data, although device face surface temperature can be used as well. The initial complex impedance values also supply information about the starting electrical conductivity of the tissue, allowing for tissue type discrimination within the neural network and potentially estimation of the type of structures (such as pockets of fat or blood vessels) near a device face. The device face surface temperature was not used due to the potential for abnormally-high interface temperatures (due to poor saline irrigation or differing densities of fat in the heterogeneous tissues, among other causes). Thus, only complex impedance values were used for the training data in the testing phase.

During testing, to collect ablation volume status data for use in calculating the ablation depth per device face, temperature probes collect tissue temperature as each ablation is run. The temperature probes (comprised of resistance temperature detectors at 0.0, 5.0, 10.0, and 15.0 mm on a carbon-fiber spine) are inserted into the target tissue at five or more points (at least one probe per margin, with the top skin-side optional for training data collection except to monitor for potential burns). The 0.0 mm sensor directly touches the surface of the ablation device 14, thus measuring the device-tissue interface temperature. The temperature collection occurs in real-time at 2 Hz, allowing for time-trigger synchronization with the complex impedance collection, which occurs whenever ablation RF current from the generator is not connected to the ablation device 14. Interpolation between the sensors allows for up sampling of temperature points for machine learning. The accuracy of this method was verified against the final gross ablation margin measured on the thermochromic tissue models.

A specific embodiment of the ablation mapping and shaping system 100 is the use of electrical complex impedance and tissue temperature data to train a machine learning classifier that can return the depth of ablation for a volume. This allows for real-time control of an ablation treatment. In addition, by abstracting complex impedance values to different tissue types and tissue statuses, treatment planning can be executed in a way that does not require strict time and power knowledge a priori.

FIG. 9 is a plot of architecture of a neural network used by the ablation mapping subsystem for providing ablation status. In traditional machine learning classifier optimization, the first step is to try a linear classifier to see if the data can be modeled as a basic linear function. A linear support vector machine (SVM) was chosen to represent the linear machine learning classifier class due to the size and type of ablation data. Since the ablation depth, impedance magnitude, impedance phase, tissue subvolume temperature and time are not linearly related, the linear SVM did not classify the status of tissue subvolumes and estimate ablation depth well. The system 100 utilizes a nonlinear machine learning classifier, specifically a multilayer perceptron (MLP) classifier, which provides improved modeling of ablation status and depth when compared to the linear SVM.

In particular, a MLP classifier is a type of artificial neural network, where incoming data (in the form of the parameters) is transformed by software neurons in hidden neuron layers to create output data, which is illustrated in FIG. 9. The estimator, when trained, will yield a computation of a nonlinear function on the weighted input sum at each neuron. Thus, if a neuron i in layer n has input weights w and input values v from the previous neuron j, and the nonlinear activation function is g, then the output value y of the neuron is:

$${y}_{i} = g ( {\sum }_{j} {w}_{i} {v}_{n - 1} )$$

Our MLP depth estimator used the rectified linear unit function for the nonlinear activation function:

g(x)=max(0, x)

Other activation functions, such as the logistic sigmoid or hyperbolic tangent functions, will yield different results. The output layer makes the final transformation of the output values of the last hidden layer to a binary classification. Training occurred via the backpropagation algorithm, which utilizes a cost function and gradient descent.

This is a form of deep learning, where machines are able to make both linear and nonlinear transformations to data in order to create classifiers with higher accuracy and precision. Features inputted into the neural network as floating-point numbers are device face, depth to estimate, initial impedance magnitude, current impedance magnitude, initial impedance phase, and current impedance phase. The output is a binary classification of whether the tissue at the face and depth specified was ablated or not. Temperature data was used to calculate the output labels for training, where tissue at 43° C.+ for ≥10 minutes, 50° C.+for ≥5 minutes, and 57° C.+ for ≥2 seconds was considered ablated. This was in accordance with existing cell death exposure models used in RFA studies

To track overfitting of the data, a training-testing data split of 70%-30% was run against training-testing data split on itself. In other words, all of the data is used to train and test. In general, a cross-validation method is used to test for overfitting in the model but given that there are over 1,000,000 support data points (the final training dataset is comprised of ˜2,000,000 data points from 72 sample ablations), a 70%-30% training-testing data split was appropriate in illustrating this modeling effect, as provided in Table 1 below:

TABLE 1
Confusion Matrix for Ablation Mapping Machine Learning Model

| Machine Type | | | |
|---|---|---|---|
| Linear SVM | | MLP Neural Network | |
| Statistical Measure | | | |
| Not ablated | Ablated | Not ablated | Ablated |
| (Code = 0) | (Code = 1) | (Code = 0) | (Code = 1) |

70% training dataset (training n = 1,266,300; testing n = 542,700)

| | | | | |
|---|---|---|---|---|
| Accuracy | 90% | 68% | 99% | 97% |
| Precision | 88% | 75% | 99% | 98% |
| True Positive Rate | 88% | 74% | 99% | 97% |
| False Positive Rate | 7% | 10% | 1% | 2% |

Full training dataset (training n = 1,809,000; testing n = 1,809,000)

| | | | | |
|---|---|---|---|---|
| Accuracy | 90% | 68% | 99% | 98% |
| Precision | 88% | 74% | 99% | 98% |
| True Positive Rate | 88% | 74% | 99% | 97% |
| False Positive Rate | 7% | 12% | 1% | 2% |

Looking at the confusion matrices shown in Table 1, there are minimal differences within each classifier trained using the split dataset and full dataset, but the important conclusion is that the MLP Neural network significantly improves upon the Linear SVM, especially for the ablated status code in both data sets. Thus, The MLP classifier is better suited to model the ablation status mapping.

Within the developed control software, the depth of ablation from the margin relative to a device face is calculated based on the depth of the deepest subvolume with an ablated status. This approach allows for error-tolerance by allowing for more estimations per device face, rather than a regression model, which only outputs one estimation of depth per device face. For example, the subvolume temperature was selected as another estimation by the machine learning classifier. Table 2, provided below, shows the results when the classifier is asked to estimate the temperature of a subvolume instead of the ablation status of a subvolume, effectively enabling the ablation device 14 to perform as a virtual tissue thermometer, if desired, as an additional functionality to the ablation status mapping. However, this would require more tuning than the ablation status mapping.

TABLE 2
Confusion Matrix for Temperature Mapping Machine
Learning Model with MLP Neural Network

| Temperature Block | | | | |
|---|---|---|---|---|
| <37° C. | 37-43° C. | 43-48° C. | 48-57° C. | >57° C. |

Full training dataset (training n = 1,809,000; testing n = 1,809,000)

| | | | | | |
|---|---|---|---|---|---|
| Accuracy | 92% | 60% | 60% | 72% | 65% |
| Precision | 88% | 67% | 66% | 69% | 71% |

To prevent interference between the LCR meter and the RF generator, the RF generator and LCR meter lines were mutually exclusive on the switch and on/off periods were alternated, as illustrated in FIG. 10. After all enabled device faces have delivered power to their local tissue for 10 seconds each, complex impedance was collected for all device faces. Temperature is collected during all times at 2 Hz, with interpolated temperatures in between 0.5 second measurement intervals.

To test the depth estimator, the ablation controller sends a request containing the collected impedance data to the depth estimator. The requests from the ablation controller software contain the data values for the features, and the real-time responses from the depth estimator software provide depth estimations for the ablation controller software. The ablation controller software will disable a device face upon the desired lesion depth per face being reached according to the depth estimation.

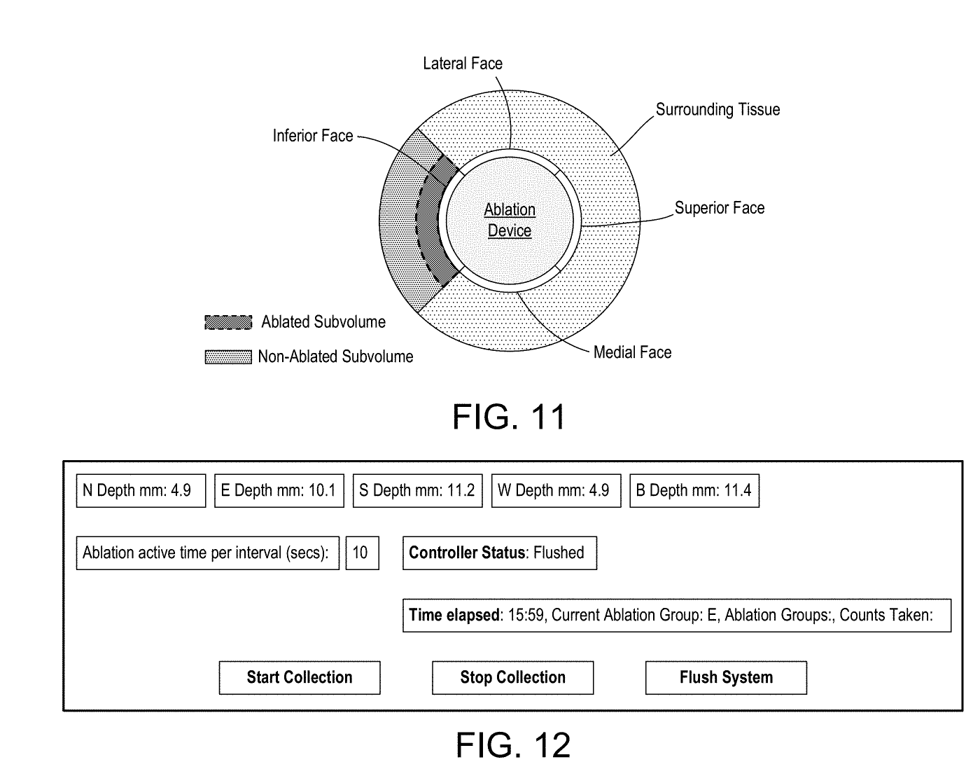

FIG. 11 is an illustration of a subvolume of target tissue relative to an activated face of the distal probing end 16 of the ablation device 14. Throughout testing, data collection and analysis was performed for each of the tissue subvolumes corresponding to the faces or sides of the distal tip 16, as previously described herein with regard to FIGS. 6A and 6B. For example, as shown in FIG. 11, the subvolume of tissue corresponding to the inferior (I) face of the device is shown, which includes an ablated subvolume portion (immediately adjacent to the I face) and a non-ablated subvolume portion. As previously described, the system 100 is configured to provide control over the RF emission from any given electrode on the device 14 is such a manner so as to accurately control depth of ablation, which can result in the ablated and non-ablated subvolumes, shown in FIG. 11.

The final electrode activation algorithm relied upon by the system 100 generally includes ablating a single device face for ten-second long durations until the desired ablation depth for the interfacing side of the target tissue margin is reached. To ablate more than one side of the target tissue margin to create the desired ablation geometry, each device face's electrodes are activated in a round-robin fashion. A uniform ablation depth (in homogeneous tissue) can be then theoretically be generated from activating all faces of the ablation device 14 device in round-robin the same number of times. For example, FIG. 12 is an exemplary output of ablation status mapping converted to ablation depth based on feedback data collected during an ablation procedure comprising electrode activation in a programmed activation algorithm. The results of this algorithm provide for very distinct separation and prevention of ablation spillage from other faces, in addition to the objective of high-resolution (millimeter-level) individual margin side ablation depths. As an added benefit, the power of RF ablation current used and deposited within the local tissue is less than previous approaches with other currently-marketed ablation devices, requiring the RF generator only be set within 30-50 W, as opposed to the 100W+ power required by currently-marketed ablation devices. This electrode activation algorithm prevents steam from occurring since the target ablation tissue does not reach phase transition temperature levels, thus clinically translating to prevention of potential skin burns or other injuries to the patient and surgeon.

Finally, regarding software architecture, there are two major components: timing of measurement cycles after ablation cycles and neural network architecture changes. The current system scans the device face impedances in sequential order, with a single device face active on positive polarity and the other device faces active on negative polarity at any given scan. However, this imposes sequentiality on the scan and ablation order. If a device face was not active during the previous ablation cycle, it may have different measurements than a device face that was active during the previous ablation cycle, simply due to the temperature of the tissue directly touching the electrode. Additionally, the system performs all ablation activations for an ablation cycle, then follows up with performing all measurement activations for a measurement cycle. This additional form of sequentiality means that tissue relaxation times (cool-down periods) between device face ablation activation and device face tissue measurement are not close or equal. Thus, the timing of the cycles is an additional limiting factor that could cause potential problems with heterogeneous tissues. Timing problems can be remedied via software, however, either by randomization or face-by-face ablate-measure cycles.

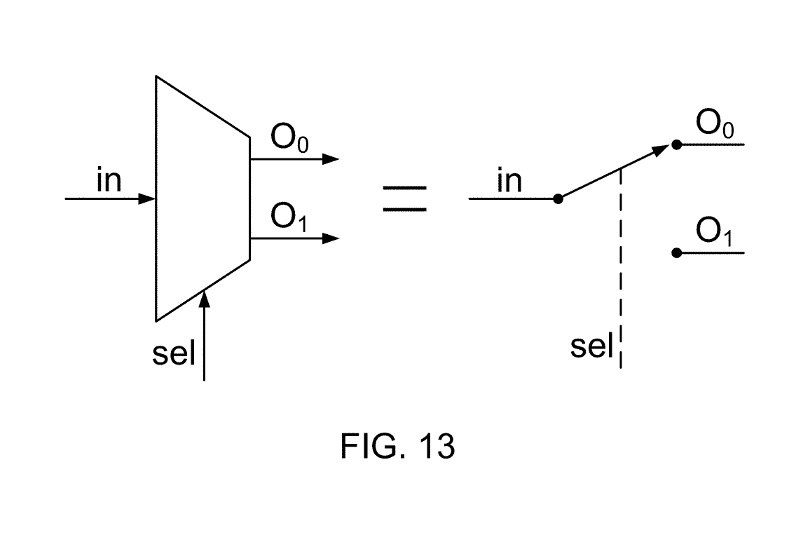

FIG. 13 is a schematic illustration of a multiplexer/switch (Mux) for use with the ablation monitoring and shaping system consistent with the present disclosure. The multiplexer/switch may be useful for allowing an input signal to be connected to outputs. The sel line is configured to determine the output lines to which the input signal will be connected. For example, as shown in FIG. 13, there are two outputs O₀ and O₁, but a Mux can include any number of outputs.

FIG. 14 is a schematic illustration of exemplary matrix switch circuitry for use with the ablation monitoring and shaping system consistent with the present disclosure. The matrix switch may be used to connect rows/columns for subsequently coupling multiple outputs to one or more inputs. For example, the matrix switch allows a single line to be switched to many different outputs, or intermediate connections, that can be indirectly or directly connected by way of the rows/columns. An input line can be connected to one or some subset of the possible output lines. For example, connections can be made to node B1C0 and B1R0 to read impedance, as shown in FIG. 14.

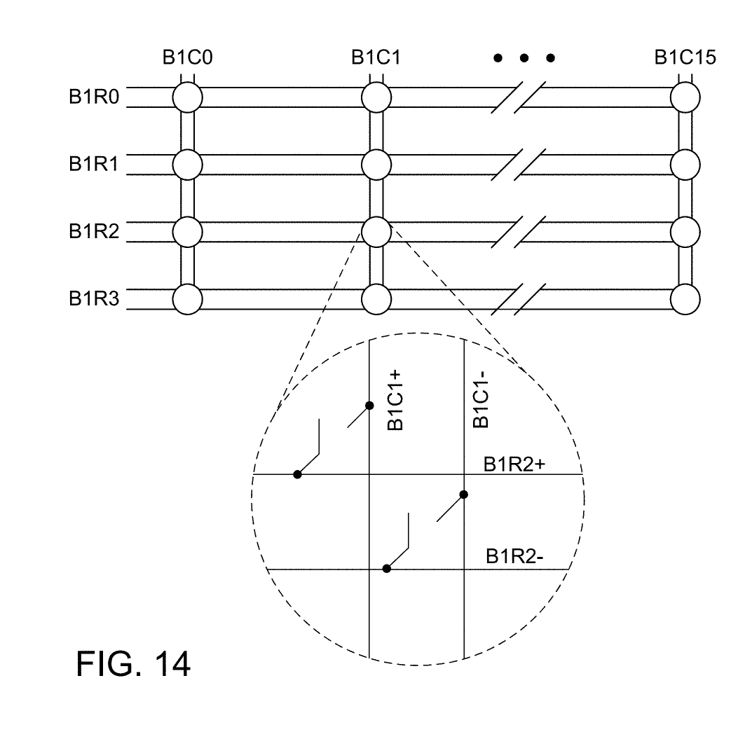

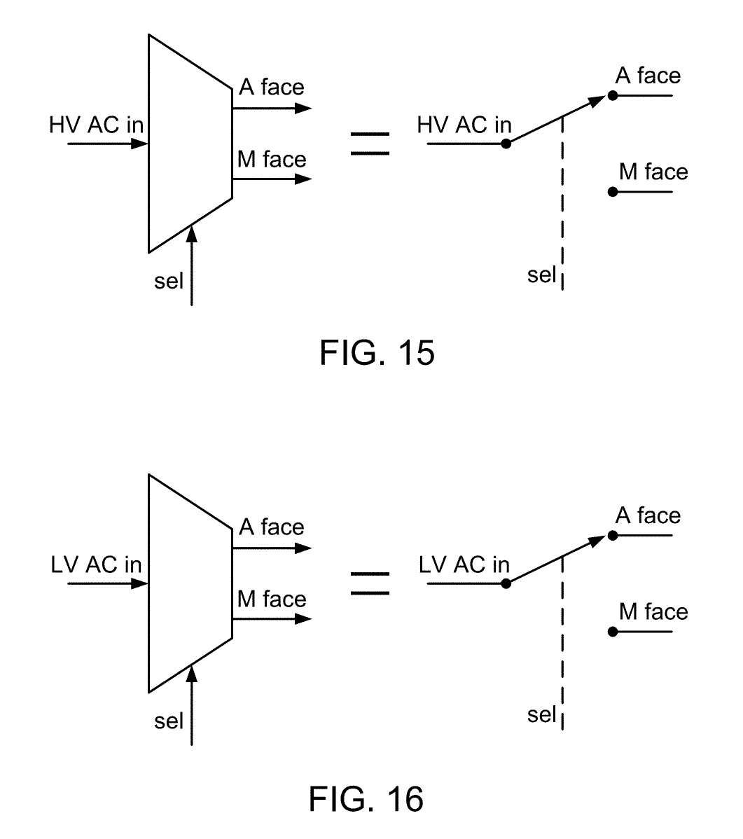

FIGS. 15 and 16 are schematic illustrations of hardware setups for different lines of multiplexers/switches (Muxes) for connecting two or more electrodes associated with different faces of the ablation device. As shown in FIG. 15, one line of Muxes includes an input, which is a high-voltage AC from an electrosurgical RF generator, and an output, which includes exposed wires (electrodes) that are positioned on a device face. As shown in FIG. 16, another line of Muxes includes an input, which is an interrogation signal (low-voltage AC) from an LCR meter, and an output, which includes exposed wires (electrodes) that are positioned on a device face.

FIG. 17 is a schematic illustration of the matrix switch circuitry of FIG. 14 for connecting lines on the ablation device. In general, the columns (B1C0, B1C1, etc.) are device faces, while rows may be used for different lines (i.e., rows B1R0 and B1R1 are used for bipolar ablation lines and rows B1R2 and B1R3 are used for an interrogation line for impedance measurements). During an ablation interval, B1R2 and B1R3 lines may be disconnected in the switch so that impedance is not measured and vice versa for ablation lines when the performing a measurement interval.

The flow process for “round robin” activation of the electrodes of the ablation device 14 is as follows: the ablation lines for a given column (a single device face) is activated for a 10 second interval; after the 10 seconds, that column is disconnected and the next column (another device face) is then connected, starting another 10 second ablation interval. A similar process is carried out for the impedance measurement using the impedance lines.

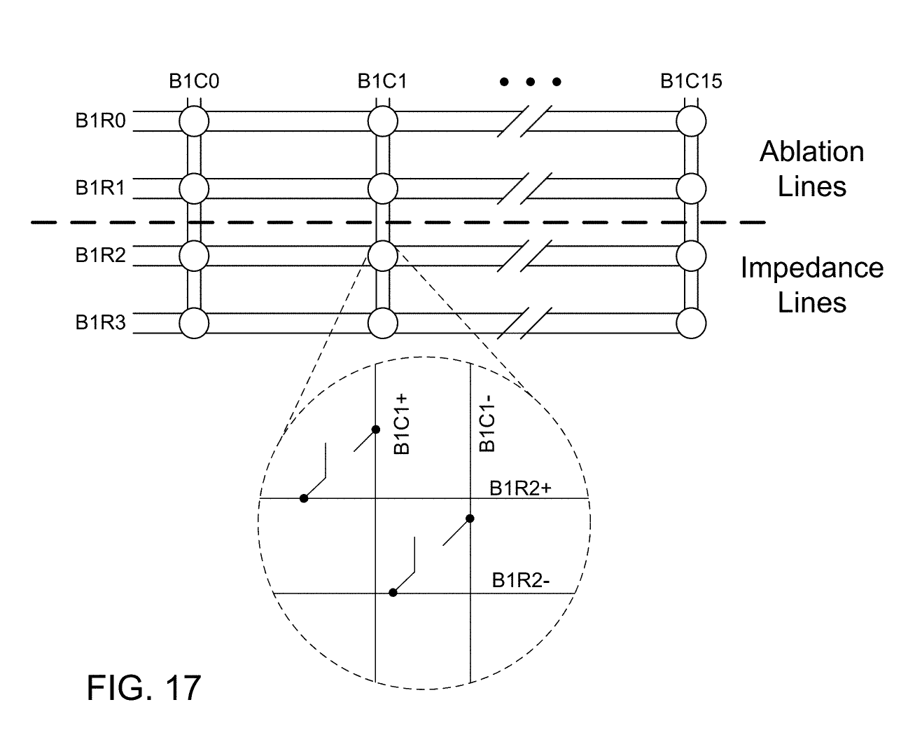

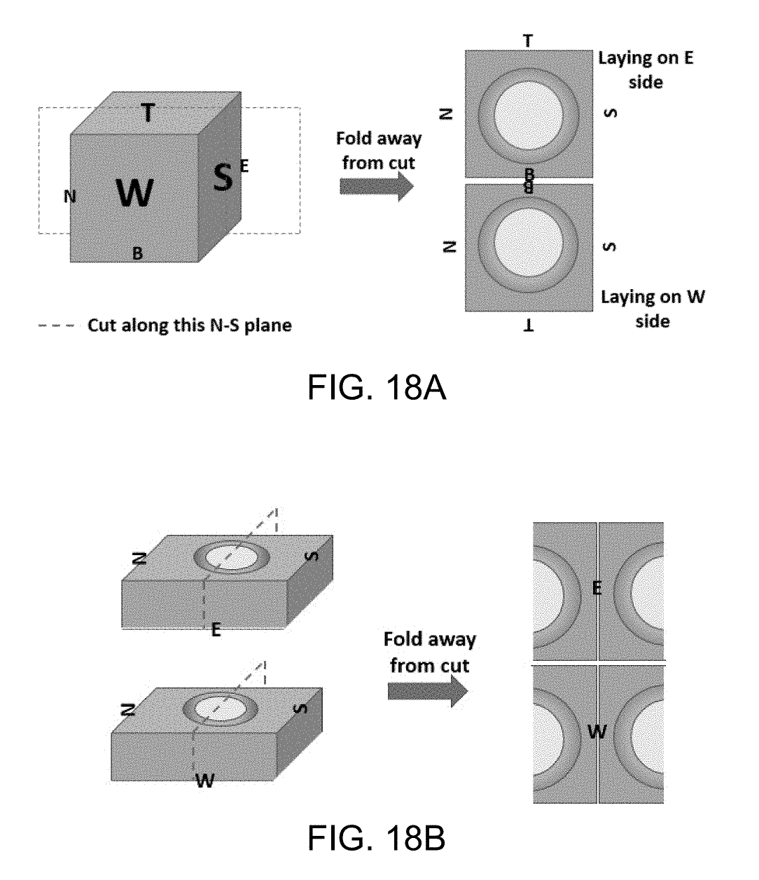

FIGS. 18A and 18B illustrate cutting methods of tissue having undergone targeted ablation using an ablation device controlled with the ablation monitoring and shaping system of the present disclosure. The cutting methods include splitting of a block of tissue into distinct portions to provide views of tissue corresponding to faces of the ablation device. In particular, the block of tissue includes a cavity in which the ablation device 14 was placed and an ablation procedure was carried out (i.e., activation of one or more electrodes resulting in ablation of targeted tissue within the cavity). The block of tissue includes a top (T), a bottom (B), and four sides designed as north (N), east (E), south (S), and west (W). Accordingly, in the scenario of performing ablation within a lumpectomy cavity in a patient's breast, the top generally corresponds to an opening of the cavity and the portion into which the device is introduced.

Thus, the top surface tissue may be thought of as a patient's skin, while the bottom may be thought of as being closing to a patient's chest cavity.

After ablation, the block of tissue is divided in half along a plane extending in a N-S direction and central between the E and W sides, as shown in FIG. 18A, thereby exposing the interior halves of the cavity. Then, each halve of the tissue block is once again divided along a plane extending in an E-W direction and central between the N and S sides, essentially now providing the tissue block in quartered sections and exposing the interior of the cavity in four different quadrants, which correspond to the superior (S), inferior (I), medial (M), and lateral (L) faces of the device 14 (as well as the anterior (A) and posterior (P) faces).

FIGS. 19A and 19B are images of a first tissue sample split into distinct portions in accordance with the cutting methods illustrated in FIGS. 18A and 18B, the images showing controlled ablation of the tissue in a targeted manner according to a first ablation pattern/geometry. As shown, only one half of the cavity has an ablated subvolume of tissue, illustrating the ability of the system 100 to control RF energy emission from the device 14 in the desired pattern to result in only half of the cavity undergoing ablation.

FIGS. 20A and 20B are images of a second tissue sample split into distinct portions in accordance with the cutting methods illustrated in FIGS. 18A and 18B, the images showing controlled ablation of the tissue in a targeted manner according to a second ablation pattern/geometry. As shown, the system 100 was able to control RF energy emission from the device 14 in such a manner so as to result in a more shallow ablation of target tissue on one half of the cavity (see depth of ablation D₁) and a deeper ablation of target tissue on the other half of the cavity (see depth of ablation D₂). By not ablating the sides of the tissue that were not selected to be ablated, the algorithm used by the system 100 is able to achieve sub-1.0 millimeter resolution. This also means avoiding ablation either completely or within 1.0 millimeter of damage of nearby critical structures a surgeon would avoid. From pulsing the sides, the electrode activation algorithm is also able to create ablation depths with sub-1.0 millimeter up to at least 10 millimeters.

The ablation depth test cases originally proposed were based on L-shaped ellipsoids, a geometry with 10 mm ablation depths for all sides except for two sides which would have 15 mm ablation depths instead. Due to interviews with breast surgeons, it was determined that ablation depths beyond 10 mm are undesirable and would rarely be used. Thus, to demonstrate the closed-loop ablation mapping and shaping system, the proposed test cases were reduced to smaller depths instead, such as 5 mm ablation depths for all sides except for two sides with 10 mm ablation depths. Table 3, provided below, lists the test cases, the number of samples, and the results.

For each ablation, the target ablation depth for each face of the ablation device was inputted into the controller software. Once the software has determined that the overall ablation is finished, we analyzed the tissue and measured the actual ablation depth for each face. Using these two measures for each face, the target depth and actual depth, we computed the mean difference, defined as the target ablation depth minus the actual ablation depth, as well as the standard error, the standard deviation divided by the square root of the number of samples. The significance of the negative mean difference of each tissue type, as well as all tissue types combined, is that the controller system errs on the side of caution and ablations more than the target ablation depth, in clinical cases, it is preferred to over-ablate the tissue containing the cancerous cells than to leave it untreated. A larger dataset to train the MLP neural network would further improve the standard error and shift the mean differences closer to 0 mm.

The systems and methods of the present disclosure can help to ensure that all microscopic disease in the local environment has been treated. This is especially true in the treatment of tumors that have a tendency to recur. Furthermore, by providing custom ablation shaping, based, at least in part, on feedback received during an ablation procedure (e.g., measured physiological parameters, including tissue impedance, temperature, and the like), the system can control a single ablation device to provide numerous RF energy emission shapes or profiles tailored to the properties (e.g., conductivity, depth, etc.) for any given target tissue. Accordingly, the system of the present invention is capable of providing optimal transmission of energy and subsequent uniform heating throughout a heterogeneous tissue during the ablation procedure. Additionally, by providing numerous RF energy emission shapes or profiles from a single ablation device, the system allows for non-uniform ablation to occur. This is particularly useful in controlling ablation shape so as to avoid vital organs and any critical internal/external structures (e.g., bone, muscle, skin) in close proximity to the tumor site, while ensuring that residual marginal tissue within the local environment has been treated.

It should be noted the devices of the present disclosure are not limited to such post-surgical treatments and, as used herein, the phrase “body cavity” may include non-surgically created cavities, such as natural body cavities and passages, such as the ureter (e.g. for prostate treatment), the uterus (e.g. for uterine ablation or fibroid treatment), fallopian tubes (e.g. for sterilization), and the like. Additionally, or alternatively, tissue ablation devices of the present disclosure may be used for the ablation of marginal tissue in various parts of the body and organs (e.g., lungs, liver, pancreas, etc.) and is not limited to treatment of breast cancer.

As used in any embodiment herein, the term “controller”, “module”, “subsystem”, or the like, may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The controller or subsystem may, collectively or individually, be embodied as circuitry that forms part of a larger system, for example, an integrated circuit (IC), system on-chip (SoC), desktop computers, laptop computers, tablet computers, servers, smart phones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

What is claimed is:

1. A system for monitoring and controlling tissue ablation, the system comprising:
a controller configured to selectively control energy emission from an electrode array of an ablation device based on ablation feedback received during an ablation procedure with the ablation device, the controller comprising a hardware processor coupled to memory containing instructions executable by the processor to cause the controller to:
receive feedback data from one or more sensors during the ablation procedure, the feedback data comprising one or more measurements associated with at least one of operation of the electrode array of the ablation device and tissue adjacent to the electrode array; and
generate an ablation pattern for controlling energy emission from the electrode array of the ablation device in response to the received feedback data.

2. The system of claim 1, wherein the measurements comprise at least one of: elapsed time during an ablation period; electrical conductivity or complex electrical impedance properties of the tissue adjacent to the electrode array; electrical conductivity or complex electrical impedance associated with one or more conductive wires of the electrode array of the ablation device; electrical current supplied to the one or more conductive wires; temperature of tissue adjacent to the electrode array; photonic properties of the tissue adjacent to the electrode array; and a combination thereof.

3. The system of claim 1, wherein the ablation pattern comprises at least one of: a selected one or more conductive wires, from a plurality of conductive wires of the electrode array, to receive electrical current for energy emission therefrom; a level of electrical current to be supplied to a selected one or more conductive wires; a length of elapsed time during which electrical current is to be supplied to a selected one or more conductive wires; one or more intervals over which electrical current is to be supplied to a selected one or more conductive wires; and a combination thereof.

4. The system of claim 3, wherein the electrode array of the ablation device comprises a plurality of independent conductive wires configured to independently receive electrical current.

5. The system of claim 4, wherein the ablation pattern comprises a selected one, or a selected set of two or more, of the plurality of conductive wires resulting in emission of energy therefrom corresponding to a portion of the electrode array, thereby resulting in targeted ablation of adjacent tissue.

6. The system of claim 1, wherein the generation of the ablation pattern comprises processing the feedback data in real-, or near-real-, time and generating ablation status mapping based on the processed feedback data.

7. The system of claim 6, wherein the ablation status mapping provides an estimation of the state of the tissue to be, currently undergoing, or having undergone ablation.

8. The system of claim 6, wherein the ablation status mapping provides an estimation of the depth of ablation of the tissue to be, currently undergoing, or having undergone ablation.

9. The system of claim 6, wherein generation of the ablation status mapping is based, at least in part, on a classification model based on training data.

10. The system of claim 9, wherein the classification model provides a plurality of reference tissue ablation parameters based on processing of the training data via a machine learning algorithm.

11. The system of claim 10, wherein the machine learning algorithm includes a nonlinear machine learning classifier.

12. The system of claim 11, wherein the classifier is a multilayer perceptron (MLP) classifier.

13. The system of claim 6, wherein generation of the ablation status mapping comprises processing the feedback data in accordance with at least one of elapsed ablation treatment time, geometric locations of tissue relative to the ablation device, initial electrical impedance between at least two electrodes of the electrode array prior to tissue ablation, initial electrical conductivity of one or more portions of tissue prior to tissue ablation, electrical impedance between at least two electrodes of the electrode array during tissue ablation, electrical conductivity of one or more portions of tissue during tissue ablation, surface temperature of one or more portions of the device, and a combination of at least two thereof.

14. The system of claim 6, wherein the generation of the ablation pattern further comprises a combination of ablation status mapping data with an electrode activation algorithm for assignment of one or more ablation control parameters for selective conductive wire activation for subsequent targeted ablation of adjacent tissue.

15. The system of claim 14, wherein the system further comprises an ablation mapping module and an ablation geometry shaping module, the ablation mapping module configured to receive and process the feedback data and transmit mapping data to the ablation geometry shaping module configured to process the mapping data to generate the ablation pattern.

16. The system of claim 15, wherein the ablation geometry shaping module is configured to transmit the ablation pattern to an electrode connection multiplexer controller configured to supply electrical current to a selected one, or set of two or more, conductive wires in response to the ablation pattern.
